(19) United States
(12) Patent Application Publication
(10) Pub. No.: US 2026/0022789 A1
(43) Pub. Date: Jan. 22, 2026

(54) BUCKLING PREVENTION FOR EAR CLAMPS

(71) Applicant: Oetiker Schweiz AZ, Horgen (CH)

(72) Inventors: Rolf DELLER, Horgen (CH); Georg KISTNER, Horgen (CH); Chris CHENG, Horgen (CH); Thomas IBER, Horgen (CH)

(73) Assignee: Oetiker Schweiz AG, Horgen (CH)

(21) Appl. No.: 19/115,886

(22) PCT Filed: Sep. 28, 2023
(86) PCT No.: PCT/EP2023/076942

§ 371 Date: Mar. 27, 2025

(30) Foreign Application Priority Data
Sep. 30, 2022 (DE) 102022125414.3

(51) Int. Cl. F16L 33/207; A61M 39/28

(52) U.S. Cl. CPC F16L 33/2071; A61M 39/28

(57) ABSTRACT

The invention relates to a band clamp including a clamping band (10) with an outer band end (12) and an inner band end (14), the outer band end (12) including at least one opening (16a-16c) and an ear-like tightening structure (18) with two radially outward extending legs (30, 32) and a web (34) interconnecting the outer ends of the legs (30, 32) and a tongue recess (20), wherein the web (34) includes a depression (36) to prevent buckling the inner band end (14) including at least one hook (22a-22c) and a tongue (28), wherein, in a pre-assembled configuration of the band clamp, the hook (22) engages in the opening (16a-16c) in the outer clamping band (10) end and a tip of the tongue (28) engages in the tongue recess (20) and the tongue (28) is configured to be moved into the togue recess when the clamp is being tightened by narrowing the ear-like tightening structure (18).

In order to reduce the risk of buckling, it is proposed that the inner band end (14) includes a longitudinal rib (24) configured to be arranged below the depression (36) of the web (34) in the pre-assembled configuration, wherein at least an end portion (24a) of said rib (24) has a height continuously decreasing towards the tongue (28) and/or dot (26) protruding toward the outer band end (12) is provided at a shoulder portion of the inner band end (14).

The invention relates to a band clamp according to the preamble of claim 1

The document U.S. Pat. No. 10,036,499B2 disclose an ear clamp configured to correspond to the tube by being sized to seal the tube against an object inserted into the tube, wherein the ear clamp comprises a clamping band including an ear-like tightening structure.

U.S. Pat. No. 3,235,925 A and U.S. Pat. No. 4,222,155 A disclose further ear clamps.

Band clamps of the claimed type risk to exhibit so-called buckling, wherein the portion of the inner band arranged underneath the ear-like tightening structure buckles rather than slipping under the outer band portion when the clamp is being tightened. In order to solve this problem, it is known to provide a depression in the web portion of the ear-like tightening structure such that the convex inner backside surface of the depression holds the band portion down when the band is being tightened.

However, while the depression reduces the risk of buckling, it cannot completely eliminate the risk. This holds in particular for medical grade clamps, which are degreased such that the surfaces exhibit larger friction coefficients, which increases the forces pushing the band to create the buckling and hence the risk of buckling. The known solutions are therefore not always satisfactory.

The invention seeks to further reduce the risk of buckling in band clamps with ear-like tightening structure.

The problem is solved by a device having the features of claim 1. Advantageous embodiments of the invention are defined in the dependent claims.

The invention relates to a band clamp including a clamping band with an outer band end and an inner band end, the outer band end including at least one opening and an ear-like tightening structure with two radially outward extending legs and a web interconnecting the outer ends of the legs and a tongue recess, wherein the web includes a depression to prevent buckling the inner band end including at least one hook and a tongue, wherein, in a pre-assembled configuration of the band clamp, the hook engages in the opening in the outer clamping band end and a tip of the tongue engages in the tongue recess and the tongue is configured to be moved into the togue recess when the clamp is being tightened by narrowing the ear-like tightening structure.

It is proposed that the inner band end includes a longitudinal rib configured to be arranged below the depression of the web in the pre-assembled configuration, wherein at least an end portion of said rib has a height continuously decreasing towards the tongue. While the rib has the twofold effect of stiffening the portion of the inner band having the rib and reducing the friction of the outer band in the course of the tightening movement of the clamp, the starting point of the rib risks to create a collision point causing buckling when the lower knee portion of one of the legs of the ear-like tightening structure moves over this point. The continuous height reduction creates a ramp facilitating a smooth slipping of the leg onto the rib.

In a preferred embodiment of the invention, the height of end portion of the rib decreases linearly over a length corresponding to at least twice a width of the rib. More preferably, the height of end portion of the rib decreases linearly over a length corresponding to at least one third of the ear-like tightening structure in the untightened configuration.

It is further proposed that the inner band end includes a shoulder portion where a width of the band is reduced from a full band width to a reduced width of the tongue, wherein a dot protruding toward the outer band end is provided at the shoulder portion, wherein a position of the centre of the dot in the longitudinal direction of the band set at a portion where the width of the band is larger than the reduced width of the tongue. The dot creates a gap between the inner band end and the outer band end at the entry portion of the tongue recess. This reduces the friction and, while the dot further stiffens the root portion of the tongue, reduces the risk of buckling.

In a preferred embodiment of the invention, a longitudinal end of the rib opposite to the end portion of the rib with decreasing height is configured to be arranged underneath one of the legs of the ear-like tightening structure in the untightened configuration.

It is further proposed that the clamping band is degreased. It has surprisingly turned out that problems with buckling can be avoided by the modifications proposed by the inventors even of the band is degreased and, as a result, the friction coefficients encountered when closing the clamp are increased.

Further features and advantages will be apparent from the following description of the embodiments and figures. The entire description, claims and figures disclose features of the invention in specific embodiments and combinations. The person skilled in the art will also consider the features individually and combine them into further combinations or sub-combinations to adapt the invention, as defined in the claims, to his needs or to specific fields of application.

The figures illustrate the following:

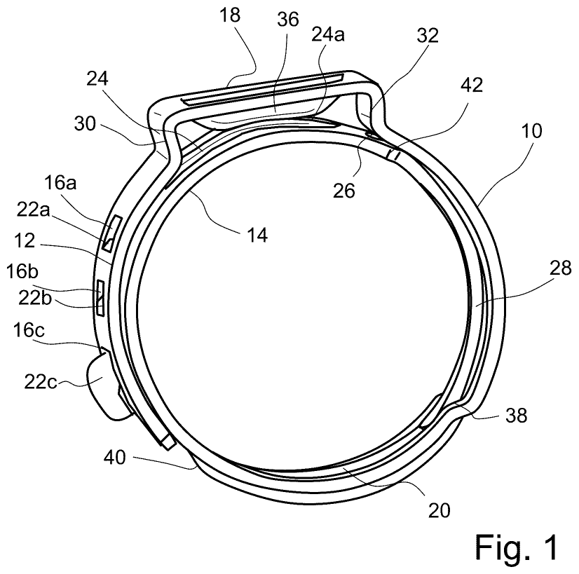

FIG. 1 a band clamp according to a first embodiment of the invention in a pre-assembled, non-tightened configuration;

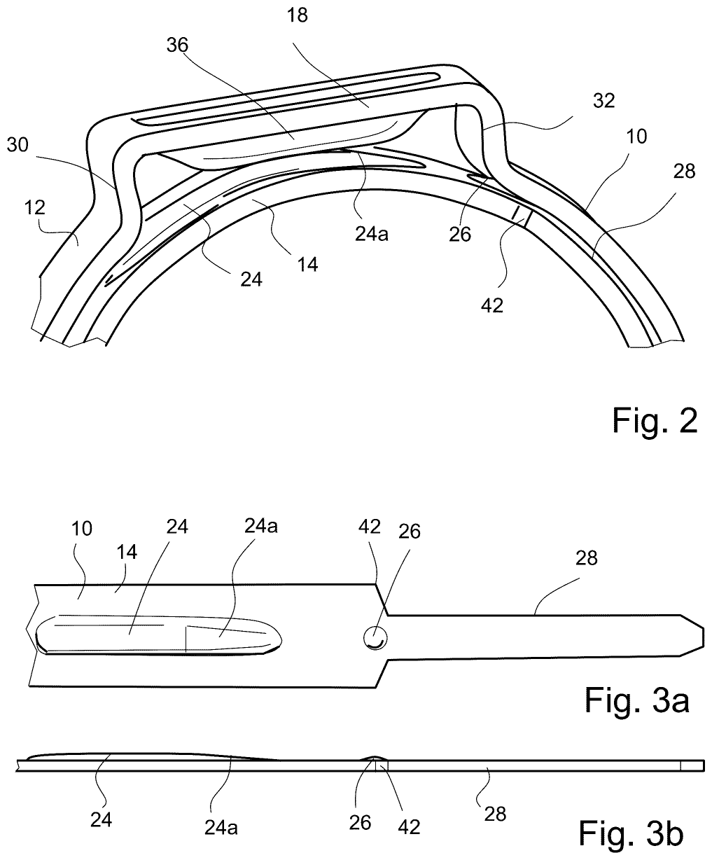

FIG. 2 a detailed view of the ear-like tightening structure of the band clamp according to FIG. 1;

FIG. 3a a top view of an inner band end of the clamp according to FIGS. 1 and 2 with a dot, and

FIG. 3b a side view of the inner band end of the clamp according to FIGS. 1 and 2.

FIGS. 1 and 2 illustrate a band clamp according to a first embodiment of the invention.

The band clamp is a de-greased medical grade clamp and includes a clamping band 10 with an outer band end 12, an inner band end 14 and a band main portion arranged between the outer band end 12 and the inner band end 14. In the order from the outer band end 12 to the inner band end 14, the following structures are provided. The outer band end 12 includes three openings 16a, 16b, 16c, an ear-like tightening structure 18 and a tongue recess 20. The inner band end 14 includes three hooks 22a, 22b, 22c, a rib 24, a dot 26 and a tongue 28.

In a pre-assembled configuration of the band clamp as illustrated in FIG. 1, the hooks 22a-22c engages in the openings 16a-16c in the outer clamping band 10 end and a tip end of the tongue 28 engages in the tongue recess 20 and the tongue 28 is configured to be moved into the tongue recess 20 when the clamp is being tightened by narrowing the ear-like tightening structure 18, e.g. using a pincer tool.

The ear-like tightening structure 18 includes two legs 30, 32 extending radially outward and a web 34 interconnecting the outer ends of the legs 30, 32 wherein the web 34 includes a depression 36 to prevent buckling.

The tongue recess 20 may be press-formed into the inner surface of the outer band end 12 or be formed by longitudinal slits (not illustrated) separating two lateral band strips from a middle portion, wherein steps 38 in the lateral band strips define an entry point of the tongue recess 20 and a step 40 in the middle potion defines an end thereof. A length of the tongue recess 20, i.e. a distance between the steps 38 at the entry points and the step 40 in the middle portion essentially corresponds to the length of the tongue 28.

According to the invention, the inner band end 14 includes the longitudinal rib 24 configured to be arranged below the depression 36 of the web 34 in the pre-assembled configuration, wherein end portion 24a of said rib 24 has a height continuously decreasing from a maximum height to zero towards the tongue 28.

More specifically, the end portion 24a of the rib 24 decreases linearly over a length corresponding more than twice a width of the rib 24, more specifically over a length corresponding to between one third and one half of the width of the ear-like tightening structure 18 in the untightened configuration, wherein the width is measured from the inner surfaces of the legs 30, 32 and corresponds to the closing stroke of the band clamp.

The inner band end 14 includes a shoulder portion 42 defining a root of the tongue 28 where a width of the band is reduced from a full band width to a reduced width of the tongue 28. A dot 26 protruding toward the outer band end 12 is provided at the shoulder portion, wherein a position of the centre of the dot 26 in the longitudinal direction of the band set at a portion where the width of the band is larger than the reduced width of the tongue 28 as illustrated in more detail in the top view of FIGS. 3a and 3b.

In the untightened configuration in FIG. 1, a longitudinal end of the rib 24 opposite to the end portion 24a of the rib 24 with decreasing height is arranged underneath the knee portion of the leg 30 of the ear-like tightening structure 18 arranged on the side of the openings 16a-16c. On the other side of the earlike tightening structure 18, the dot 26 is arranged underneath the knee portion of the other leg 32. Both the rib 24 and the dot 26 create small gaps between the outer band end 12 and the inner band end 14 on both sides of the ear-like tightening structure 18. The gaps reduce the friction, in particular at the shoulder portion of the tongue 28, in the course of the closing movement. The reduced friction prevents buckling of the band, in particular bucking of the portion of the inner band arranged between the legs 30, 32 of the ear-like tightening structure 18. The effect of buckling prevention is further strengthened by the stiffening effect of the rib 24 provided in that portion and further by the inclined ramp at the end portion 24a thereof.

When the clamp is being tightened, the tongue 28 moves in the tongue recess 20 and the knee portion of the leg 32 arranged on the side of the tongue 28 slips over the ramp with increasing height on the end portion 24a of the rib 24. The depression 36 on the web 34 of the ear-like tightening structure 18 may come in contact with the rib 24 and prevent bucking of the portion of the band underneath the ear-like tightening structure 18 in the event that such bucking happens despite the above additional measures for reducing the friction.

The invention is not limited to the embodiment described above but may be applied to other kinds of clamps, e.g. clamps with longer closing stroke having two depressions 36 on the web 34 of the ear-like tightening structure 18 or to clamps. Moreover, it would be possible to provide two ribs 24 and/or two dots 26 adjacent to each other in a width direction of the band. Non-uniform height structures of the depressions 36 could be combined with a non-uniform height profile of the rib 24, wherein a deeper portion of the depression 36 could be paired with a shallower portion of the A further embodiment of the invention includes only a dot 26 at the root portion of the tongue 28 and no rib 24 underneath the ear-like tightening structure 18.

### LIST OF REFERENCE NUMBERS

10 clamping band
12 outer band end
14 inner band end
16 opening
18 ear-like tightening structure
20 tongue recess
22 hook
24 rib
24a end portion
26 dot
28 tongue
30 leg
32 leg
34 web
36 depression
38 step
40 step
42 shoulder portion

1. A band clamp including a clamping band (10) with an outer band end (12) and an inner band end (14), the outer band end (12) including at least one opening (16a-16c) and an ear-like tightening structure (18) with two radially outward extending legs (30, 32) and a web (34) interconnecting the outer ends of the legs (30, 32) and a tongue recess (20), wherein the web (34) includes a depression (36) to prevent buckling the inner band end (14) including at least one hook (22a-22c) and a tongue (28), wherein, in a pre-assembled configuration of the band clamp, the at least one hook (22a-22c) engages in the at least one recess (16a-16c) in the outer clamping band (10) end and a tip of the tongue (28) engages in the tongue recess (20) and the tongue (28) is configured to be moved into the togue recess when the clamp is being tightened by narrowing the ear-like tightening structure (18), wherein the inner band end (14) includes a longitudinal rib (24) configured to be arranged below the depression (36) of the web (34) in the pre-assembled configuration, wherein at least an end portion (24a) of said rib (24) has a height continuously decreasing towards the tongue (28).

2. The band clamp according to claim 1, wherein the height of the end portion (24a) of the rib (24) decreases linearly over a length corresponding to at least twice a width of the rib (24).

3. The band clamp according to claim 1, wherein the height of the end portion (24a) of the rib (24) decreases linearly over a length corresponding to at least one third of the ear-like tightening structure (18) in the untightened configuration.

4. The band clamp according to claim 1, wherein the inner band end (14) includes a shoulder portion where a width of the band is reduced from a full band width to a reduced width of the tongue (28), wherein a dot (26) protruding toward the outer band end (12) is provided at the shoulder portion, wherein a position of the center of the dot (26) in the longitudinal direction of the band set at a portion where the width of the band is larger than the reduced width of the tongue (28).

5. The band clamp according to claim 1, wherein a longitudinal end of the rib (24) opposite to the end portion (24a) of the rib (24) with decreasing height is configured to be arranged underneath one of the legs (30, 32) of the ear-like tightening structure (18) in the untightened configuration.

6. The band clamp according to claim 1, wherein the clamping band (10) is degreased.
